(19) United States
(12) Patent Application Publication
(10) Pub. No.: US 2014/0012152 A1
(43) Pub. Date: Jan. 9, 2014

(54) Method of Decreasing Sensory Latency

(71) Applicant: NystAssist, LLC, Gastonia, NC (US)

(72) Inventors: John R. Gentry, Gastonia, NC (US); Rachael Anne Wruble, Gastonia, NC (US); Raymond Terry Riddle, Middleton, WI (US); Betholyn F. Gentry, Little Rock, AR (US)

(73) Assignee: NystAssist, LLC, Gastonia, NC (US)

(21) Appl. No.: 13/720,104

(22) Filed: Dec. 19, 2012

Related U.S. Application Data
(60) Provisional application No. 61/577,381, filed on Dec. 19, 2011.
(60) Provisional application No. 61/598,720, filed on Feb. 14, 2012.

(51) Int. Cl. A61H 39/02; A61B 5/00; A61H 39/04; A61B 5/0484; A61B 5/048

(52) U.S. Cl. CPC A61H 39/02; A61B 5/0484; A61B 5/048; A61H 39/04; A61B 5/4836
USPC 600/544; 606/204

(57) ABSTRACT

Localized persistent tactile stimulation provides a decreased sensory latency in response to sensory stimulation leading to a mechanism of treating nystagmus and possibly dyslexia and autism by locating the tactile stimulation under guidance of multiple measurements of sensory latency.

### STATEMENT REGARDING FEDERALLY SPONSORED RESEARCH OR DEVELOPMENT

### CROSS REFERENCE TO RELATED APPLICATION

This application claims the benefit of U.S. provisional application 61/577,381 filed Dec. 19, 2011 and U.S. provisional application 61/598,720 filed Feb. 14, 2012 both hereby incorporated in their entirety by reference.

### BACKGROUND OF THE INVENTION

The present invention relates to a method and apparatus for reducing sensory latency such as may underlie certain conditions of nystagmus, dyslexia, attention deficit, hyperactivity and autism.

In order to see a clear image an individual needs to hold the line of sight steady. Congenital nystagmus is a pathologic oculomotor state of involuntary horizontal eye movements that interferes with this steady line of sight thereby reducing visual acuity. Generally, congenital nystagmus has been considered untreatable although some drugs exist that may be effective against nystagmus for certain individuals. Some studies suggest that acupuncture may be effective against the symptoms of nystagmus when applied on the neck muscles (sternocleidomastoid).

### SUMMARY OF THE INVENTION

The present invention provides a method of reducing sensory latency, that is, the delay between sensory stimulus, such as a change in visual pattern, and evoked brain activity as evidenced by EEG measurements. This reduced sensory latency is obtained by long-term local tactile stimulation, for example, by common acupressure beads. The technique appears to provide substantial relief from nystagmus and may provide beneficial effects for the treatment of other disorders linked to sensory latency including dyslexia, attention deficit disorder, hyperactivity and autism.
Specifically, the present invention provides a method of decreasing sensory latency comprising the steps of: (a) obtaining a baseline reading of sensory evoked potential of the patient; (b) applying at least one source of persistent tactile stimulation to the patient;(c) obtaining a subsequent reading of sensory evoked potential of the patient to evaluate change in sensory latency; and (d) repeating steps (b) and (c) after adjusting a location of at least one source of persistent tactile stimulation to decrease the sensory latency.

It is thus a feature of at least one embodiment of the invention to provide a systematic method of treating conditions having an underlying cause of excessive sensory latency including nystagmus, dyslexia, and autism.

The reading of the sensory evoked potential may be a reading of visual evoked potential triggered by a visual display.

It is thus a feature of at least one embodiment of the invention to make use of existing visual evoked potential equipment for implementing and optimizing the present method.

The sources of persistent tactile stimulation may be at least one bead attached to the patient's skin to press against the skin.

It is thus a feature of at least one embodiment of the invention to provide a method that may make use of readily available acupressure beads for the tactile stimulation.

The beads may include an overlying adhesive backing material adhering to the skin at a periphery of the material and holding a bead toward the center of the material.

It is thus a feature of at least one embodiment of the invention provide for a multi-day tactile stimulation with a relatively simple and intuitive mechanism.

The overlying adhesive backed material may hold multiple spaced apart beads. It is thus a feature of at least one embodiment of the invention to provide a simple method of producing repeatable multipoint tactile stimulation.

The persistent tactile stimulation may be provided in regions from a group consisting of the patient's face, the inside of the patient's wrist, the front of the patient's lower leg, and the region of the patient's ears.

It is thus a feature of at least one embodiment of the invention to locate the beads in positions that may provide increased efficacy.

The method may obtain a frequency domain transformation of the sensory evoked potential to compare spectral power in a low and relatively higher frequency band to deduce a degree of sensory latency.

It is thus a feature of at least one embodiment of the invention to provide for a quantification of sensory latency that may be used to assess location of the beads and effectiveness of the treatment.

The low frequency band may be substantially 5 to 10 hertz and the upper frequency band is substantially 16 to 20 hertz.

It is thus a feature of at least one embodiment of the invention to perform frequency measurements in bands that conform to theta and beta EEG waveforms.

More specifically, the present invention provides a method of treating nystagmus comprising the step of applying a persistent tactile stimulation to patient regions from the group consisting of the patient's face, the inside of the patient's wrist, the front of the patient's lower leg, and the region of the patient's ears over a multi-day period.

It is thus a feature of at least one embodiment of the invention to provide a drug-free treatment for nystagmus.

These particular objects and advantages may apply to only some embodiments falling within the claims and thus do not define the scope of the invention.

### BRIEF DESCRIPTION OF THE DRAWINGS

FIG. 1 is a simplified perspective view of a patient monitored with a visual evoked potential machine for measuring visual evoked potential;

FIG. 2 is a plot of EEG activity measured by the machine of FIG. 1 with respect to time after a visual event showing two curves of EEG activity, with and without treatment by the method of the present invention;

FIG. 3 is a Fourier transform of the curves of FIG. 2 showing bands of energy measurement for creation of a theta beta ratio;

FIG. 4 is a cross-sectional view of an acupressure bead pressed against the skin as may be used in the present invention;

FIG. 5 is a perspective view of the acupressure bead of FIG. 4 as applied to the skin;

FIG. 6 is a detail view of a human wrist showing one possible placement of beads for the present invention;

FIG. 7 is a detail view of a human ear showing three possible locations of beads for the present invention;

FIG. 5 is a detail view of a patient's lower leg showing an alternative or additional location of beads per the present invention;

FIG. 9 is a front elevational view of a human face showing additional or alternative locations for the beads of the present invention;

FIG. 10 is a perspective view of glasses adapted to provide for behind the ear tactile stimulation such as may be useful in the present invention; and

FIG. 11 is a simplified flowchart of the principal steps of the present invention.

### DETAILED DESCRIPTION OF THE PREFERRED EMBODIMENT

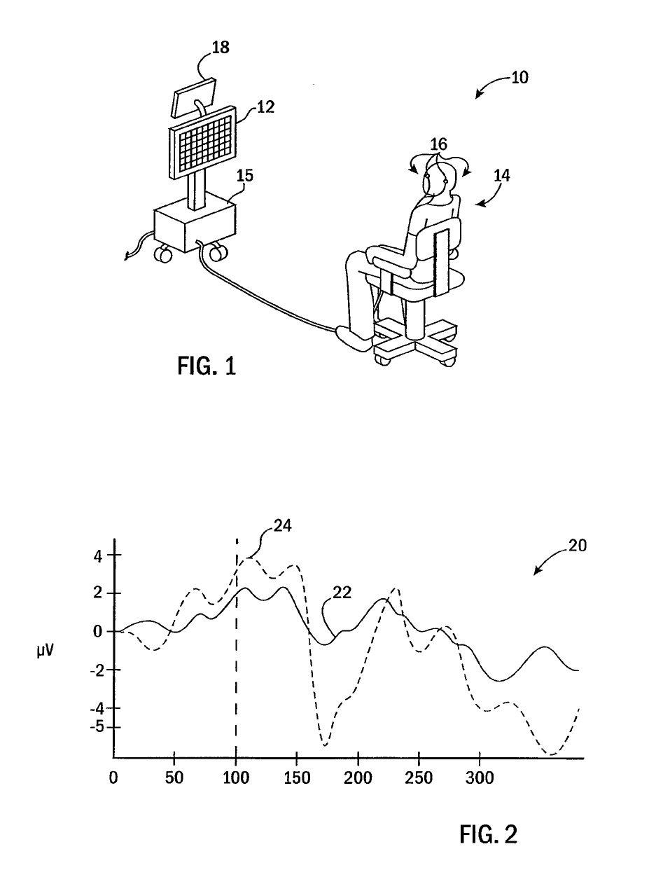

Referring now to FIG. 1, an oculomotor testing system 10 may provide for a graphics display screen 12 supported to face toward a seated patient 14 with the display screen 12 at approximately head height of the seated patient 14. The display screen 12 may provide for a varying visual display under the control of a computer system 15, for example, the visual display being a checkerboard pattern switching to its negative image at an event time. EEG signals may be obtained from electrodes 16 placed on the front, left and right side, and rear of the patient's head to collect EEG signals from the patient 14. These EEG signals may be aligned to the event time and displayed on a diagnostic display 18 to a clinician as will be discussed below. It will be appreciated that computer system 15 provides for standard EEG processing circuitry as well as a general purpose computer processor executing a stored program whose behavior will be described below held in memory.

Referring now to also to FIG. 2, the diagnostic display 18 may display an EEG activity graph 20 providing stored and latest data in the form of a baseline curve 22 and a latest curve 24, both showing EEG activity by a same patient aligned to the event time. Generally, the baseline curve 22 represents generally EEG activity before treatment or before a change in treatment by the method of the present invention and the latest curve 24 represents generally current EEG measurements after a predetermined length of treatment by the method of the present invention. Both curves 22 and 24 reflect EEG activity as aligned to a time zero being the event time of the change of display on the display screen 12 described above. Generally, successful treatment by the method of the present invention will produce substantially greater variability in latest curve 24 with respect to baseline curve 22, the greater variability equating to decreased sensory latency.

An oculomotor testing system 10 of a type suitable for the present invention is commercially available from Diopsys of Pinebrook, N.J. and is described in multiple U.S. patents including Pat. Nos.: 6,475,162; 7,578,795; 8,083,354 and 8,100,533 hereby incorporated by reference.

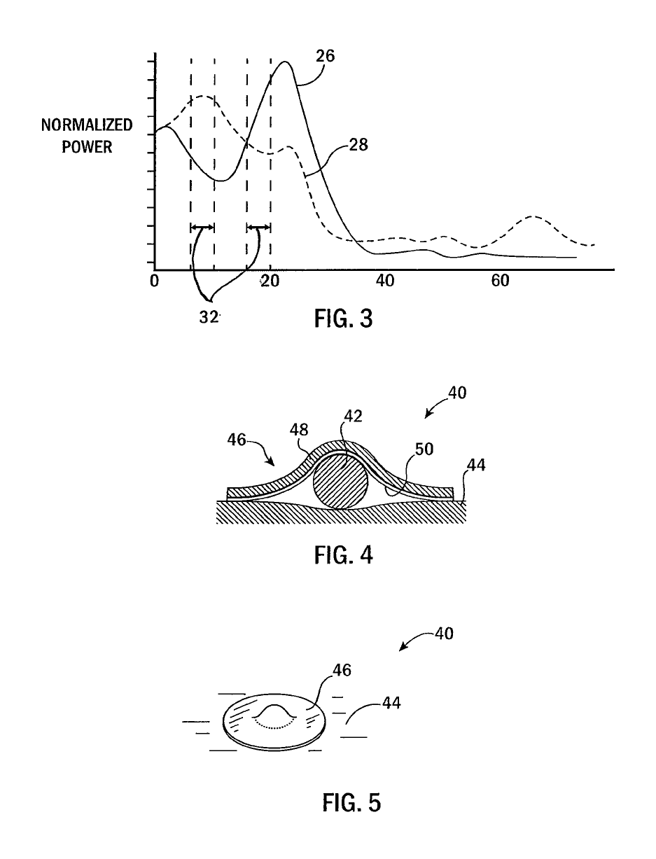

Referring now also to FIG. 3, a Fourier transform of curves 22 and 24 may be performed to provide spectral curves 26 and 28, respectively. As is understood in the art, a Fourier transform produces a frequency spectra, in this case an EEG power versus frequency in hertz. The power spectra may be divided into a theta band 30 of frequency approximately 7 to 10 hertz and a beta band 32 of frequency from approximately 16 to 20 hertz. A ratio of the integral of power within these bands (for example for the first 100 ms of curves 22 and 24) provides a theta beta ratio presenting a quantifiable indication of sensory latency. In the example of FIG. 3, for curve 26 being based on baseline curve 22 before treatment by the present invention, the theta beta ratio is 4.1. In contrast, for the curve 28 based on curve 24, the theta beta ratio is 2.25. Generally the present invention will endeavor to locate tactile stimuli on the patient to reduce sensory latency and to lower the theta beta range to a practical minimum and in any event less than three.

Referring now to FIGS. 4 and 5, a convenient source of tactile stimulation is acupressure patches 40 providing, for example, a small magnetic or un-magnetized spherical metal bead 42 (for example stainless steel, gold plated steel, and titanium) typically greater than one millimeter in diameter and often from 1.5 to 2 millimeters in diameter and preferably at least two millimeters in diameter. The bead 42 or multiple beads 42 may be held against the skin 44 by means of an adhesive patch 46 providing, for example, a flexible vinyl or fabric backing layer 48 having an adhesive 50 placed on its lower side toward the skin 44 to hold the bead 42 against the skin 44 in the manner of an adhesive bandage. Pressure by the backing layer 48 on the bead 42, periodic pressing on the bead 42 by the patient, and inertial forces on the bead 42 provide tactile stimulation at the location of the bead 42. Beads of this type are generally available in the commercial market for other acupressure purposes, for example, from Lhasa OMS, Inc. of Weymouth, Mass.

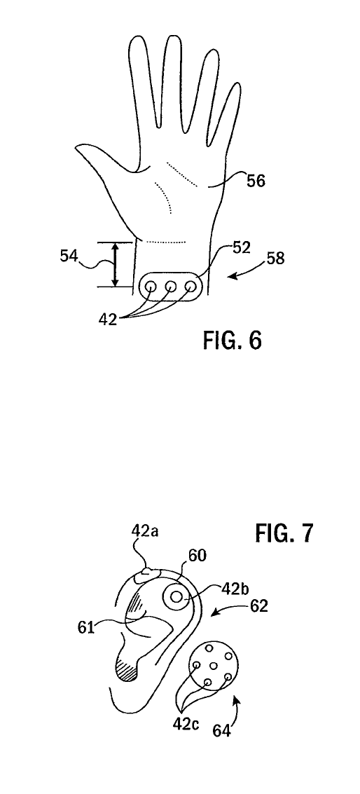

Referring now to FIGS. 6-9, the present inventors have identified a number of locations for placement of the acupressure patches 40 that appear to provide efficacy in the present method although this list is not intended to be exhaustive. As shown in FIG. 6, a patch 52 of three beads 42 may be placed at a distance 54 approximately three finger widths beneath the hand 56 on the inside of the wrist 58 extending across the wrist 58.

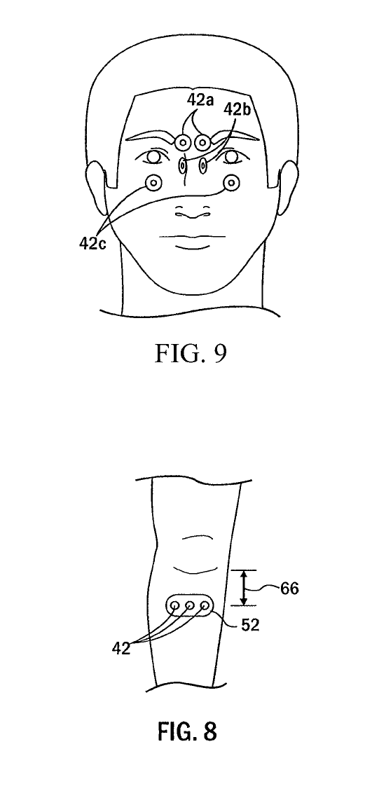

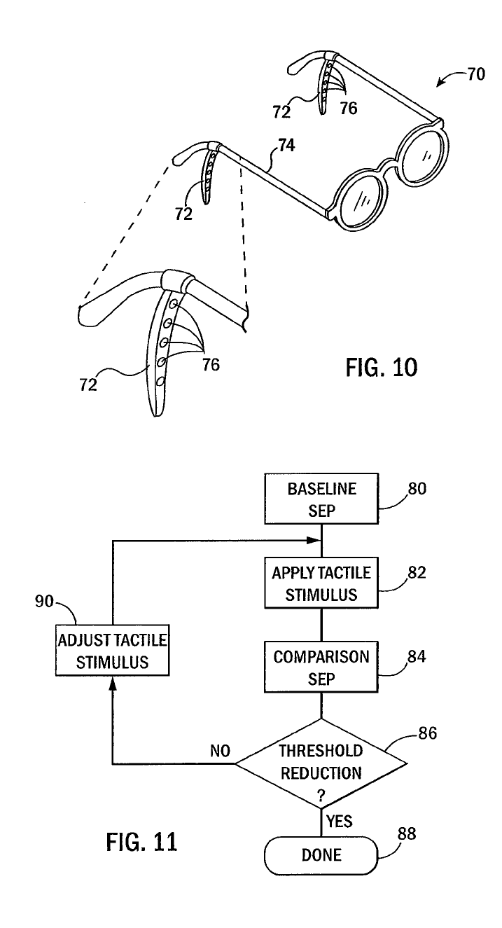

Referring to FIG. 7, individual beads 42 may be placed at an apex of the outer ear helix 60 (as shown by bead 42a) or in the pocket 61 of the ear 62 (scapha) directly below that point of bead 42a or bead 42b or in a cluster of five beads 42c on the scalp 64 behind the ear 62.

Referring to FIG. 8, a patch 52 holding three beads 42 and similar to the patch described with respect to FIG. 6 may be placed on the front of the patient's leg below the knee by a distance 66 of approximately four finger widths.

Referring to FIG. 9, bilaterally symmetric pairs of beads 42a may be placed on the bony ridge between a patient's eyebrows, or between the patient's eyes on either side of the bridge of the nose as indicated by beads 42b or on the bony ridge beneath the patient's eyes above the nostrils as indicated by beads 42c.

Referring now to FIG. 10, long-term tactile stimulation in the region of the ear may be provided by glasses 70 having added downward extensions 72 from the earpieces 74 of the glasses 70, so that the downward extensions fit tightly against a rear surface of the outer ear (not shown). The front of these downward extensions 72 may be studded bead-like projections 76 to provide tactile stimulation against the outer surface of the rear of the individuals ears. A general framework for the downward extensions 72 may be provided by a Croakies™ Reax Device commercially available from Croakies of Bozeman, Mont.

Referring now to FIG. 11, the present invention provides for measurement of a baseline sensory evoked potential as indicated by process block 80 prior to treatment. At succeeding process block 82 tactile stimulation is applied to the patient's skin in particular locations, for example, selected from those described above. The benefit of the present invention appears to be lost when the stimulation ends and accordingly the stimulation is preferably substantially continuous over 24 hours.

After a period of time, for example, at least 24 hours, a comparison sensory evoked potential measurement may be made as indicated by process block 84. Improvements in threshold latency may be determined as indicated by decision block 86 between the baseline measurement of process block 80 and the latest comparison sensory evoked potential measurement of process block 84. If a threshold level of decreased sensory latency has been reached or a minimum predetermined number of iterations in adjusting the position of this tactile stimulation, the process is done as indicated by process block 88. Otherwise, as indicated by process block 90, the location and/or number of the tactile stimulation locations is changed or increased and the process loops back to process block 84 for a new comparison between the previous measurement at process block 82 and the latest measurements at process block 82. It will be appreciated that in the event of an increase in sensory latency, comparisons with the previous best decrease in sensory latency will be used in a hill climbing fashion.

While it will be appreciated that clinical evaluation of nystagmus symptoms per process block 84 with respect to location of the beads is desirable, the invention also contemplates that the acupressure patches 40 may be located and adjusted in a home kit providing the acupressure patches 40 and instructions for trying different locations for the acupressure patches 40 based on periodic self-assessment of improvement in nystagmus symptoms, for example, using an ability to track moving dots provided on a computer display or prerecorded video program.

In one embodiment, the oculomotor testing system 10 may be combined with automatic electrical stimulation provided by low-power electrical signals applied to the skin in multiple locations. Electrical stimulation allows machine control of process block 82 permitting semiautomatic identification of stimulation points by cycling through different stimulation points while measuring sensory evoked potentials to minimize sensory latency.

In one embodiment, the oculomotor testing system 10 may be implemented in a low-cost version through the use of commercial brain monitors such as are available commercially under the tradename NeuroSky Mindset or Mindwave EEG monitors from NeuroSky of San Jose Calif. as well as other vendors. These devices provide an output that directly indicates amplitude of standard brainwave bands including the band of theta waves and beta waves that may be used directly calculate the theta beta ratio for an individual as described above. Alternatively, the raw EEG signals may be processed directly, also using the techniques described described above, or after spectral analysis also provided by devices themselves as a standard feature. The use of a commercial brainwave monitor of this type significantly lowers the cost of this system allowing it to be practical for home use. It is believed that a general evaluation of the EEG signals in any of the above-described methods may provide for the necessary guidance with regard to the location of the tactile stimuli without the need for synchronized visual or audio stimulation.

The present invention also contemplates that it may be used with other sensory evoked potential measuring systems, for example those that substitute an audio signal for the visual signal provided by the oculomotor testing system 10.

It is believed that the invention may be applied to other disorders related to sensory latency, for example, attention deficit disorders as noted in: Yordanova J, Heinrich H, Kolev V, Rothenberger A, “Increased event-related theta activity as a psychophysiological marker of comorbidity in children with tics and attention-deficit/hyperactivity disorders”, Neuroimage. 2006 Aug 15; 32(2): 940-55, Epub (2006); and Oades R D, Dittmann-Balcar A, Schepker R, Eggers C, Zerbin D, “Auditory event-related potentials (ERPs) and mismatch negativity (MMN) in healthy children and those with attention-deficit or tourette/tic symptoms”, Biol Psychol. (1996) 12; 43(2):163-85.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

References to a computer can be understood to include one or more microprocessors that can communicate in a stand-alone and/or a distributed environment(s), and can thus be configured to communicate via wired or wireless communications with other processors, where such one or more processor can be configured to operate on one or more processor-controlled devices that can be similar or different devices. Furthermore, references to memory, unless otherwise specified, can include one or more processor-readable and accessible memory elements and/or components that can be internal to the processor-controlled device, external to the processor-controlled device, and can be accessed via a wired or wireless network.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties.

What we claim is:

1. A method of decreasing sensory latency comprising the steps of:
(a) obtaining a baseline reading of sensory evoked potential of a patient;
(b) applying at least one source of persistent tactile stimulation to the patient;
(c) obtaining a subsequent reading of sensory evoked potential of the patient to evaluate change in sensory latency; and
(d) repeating steps (b) and (c) after adjusting a location of the at least one source of persistent tactile stimulation to decrease the sensory latency.

2. The method of claim 1 wherein the reading of sensory evoked potential is visual evoked potential.

3. The method of claim 1 wherein at least one source of persistent tactile stimulation is at least one bead attached to the patient's skin to press against the skin.

4. The method of claim 3 wherein each bead includes an overlying adhesive backing material adhering to the skin at a periphery of the material and holding the bead within a periphery of the material.

5. The method of claim 4 wherein the overlying adhesive backed material holds multiple spaced apart beads.

6. The method of claim 3 wherein the beads are metal beads of a diameter of at least one millimeter.

7. The method of claim 1 wherein the persistent tactile stimulation is provided in regions selected from a group of: the patient's face, an inside of the patient's wrist, a front of the patient's lower leg, and a region of the patient's ears.

8. The method of claim 1 further including the step of obtaining a frequency domain transformation of the sensory evoked potential to compare spectral power in a low and relatively higher frequency band to deduce a degree of sensory latency.

9. The method of claim 8 wherein the low frequency band is substantially 5 to 10 hertz and the relatively higher frequency band is substantially 16 to 20 hertz.

10. The method of claim 9 wherein the low frequency band is substantially 5 to 10 hertz and the upper frequency band is substantially 16 to 20 hertz.

11. A method of treating nystagmus comprising the step of applying a persistent tactile stimulation to patient regions from the group consisting of the patient's face, an inside of the patient's wrist, a front of the patient's lower leg, and a region of the patient's ears over a multi-day period.

12. A kit for a treatment of nystagmus comprising:
a set of pressure providing beads include an overlying adhesive backing material adhering to skin at a periphery of the material and holding a bead toward a center of the material; and
an instruction brochure describing an assessment of nystagmus symptoms, potential location points of the pressure-providing beads, and an evaluation of those location points based on the assessment of nystagmus symptoms.
